(19) United States
(12) Patent Application Publication
(10) Pub. No.: US 2004/0249308 A1
(43) Pub. Date: Dec. 9, 2004

(54) Sampling device

(76) Inventor: Jan Forssell, Parainen (FI)

Correspondence Address: COHEN, PONTANI, LIEBERMAN & PAVANE, 551 FIFTH AVENUE, SUITE 1210, NEW YORK, NY, 10176, US

(21) Appl. No.: 10/491,387

(22) PCT Filed: Aug. 19, 2002
(86) PCT No.: PCT/FI02/00679

(30) Foreign Application Priority Data
Oct. 1, 2001 (FI) 20011918

(51) Int. Cl. A61B 5/00

(52) U.S. Cl. 600/573

(57) ABSTRACT

The invention relates to a method for sampling body fluid of a living being by using a sampling device, which obtains a sample that is led outside a skin or the like surface of a living being by a catheter, cannula or the like, the sample being led via a tube directly or to be conveyed to an analysing device or to be otherwise examined in order to define at least partly the contents of the body fluid. A transfer pump suctions the sample brought outside a skin or the like surface towards the analysing device through a transfer tube. The sample being of a sufficient size, a sampling passage to a catheter, cannula or the like of the target to be examined is closed with a shutter, after which at least one transport medium is led into the tube behind the sample.

The invention relates to a method according to the preamble of the independent claim 1 and to a sampling device according to the independent claim 5 for sampling body fluid of a living being as well as to a method according to the independent claim 12 for transferring infusion fluid into a living being via a catheter, cannula or the like.

The traditional way e.g. for drawing a blood sample is to use a hollow tube inserted into a vessel, along which tube blood can run into a test tube. This is meant to be drawn only occasionally, because it is necessary to make an intravenous connection for every sampling. It is known to use a catheter, cannula or the like in such a manner that e.g. nutrient solution or solution containing a drug is transferred via it into a living being. Such a device usually also has a capability for sampling, most usually for blood sampling. Mostly samples are taken to a laboratory where they are examined. This has been found difficult, because it involves transports at least within the hospital. Nowadays, also an analysing unit is used, to which the sample is transferred as a so-called close transfer, that is, the analysing unit (e.g. of the type Philips CMS 2002 Blood Analysis Portal) is close to the sickbed, operating table or the like. Even then, the sample is to be taken in some way at least partly manually and to be transferred to the said analysing unit of the sample. The said equipment is connectable to a patient monitor, which usually monitors the blood pressure, pulse, EKG-diagram showing the function of the heart and also other electrophysiological parameters of the patient.

Publication U.S. Pat. No. 5,758,643 describes a sampling device, in which body fluid is taken from a living being but the body fluid is pumped back after analysing. Here it is possible that the body fluid to be pumped back could disadvantageously change because of analysing, or that it might catch some harmful substances, which could be even fatal. The analysing equipment described is also defective in some respect. Publication U.S. Pat. No. 4,974,592 describes one approach to sampling, but flushing is effected with heparin solution, which is led to a patient. The equipment is also remarkably complicated. Publication GB 2194641 describes a sampling arrangement in which blood is re-infused to the patient after examination, which can be harmful or dangerous.

The object of the invention is to describe a method and a sampling device for sampling body fluid of a living being as well as a method for transferring infusion fluid into a living being via a catheter, cannula or the like, having such a structure, that the size of a single sample would be as small as possible but sufficient for carrying out an analysis. A further object of the invention is to separate the sample that is in the tube with transport medium, air or inert gas and/or transfer fluid, so that there is transport medium on both ends of the sample. A basic object of the invention is to automate sampling, so that the sampling event would be carried out as automatically as possible with the method and equipment according to the invention, even at timed regular intervals that can be set, and in addition whenever needed, for example triggered by changes in the physiological parameters of the target. A further object of the invention is to present a possibility to perform regular infusion when sampling is not being carried out.

The object of the invention is achieved by what is presented in the method according to the independent claim 1, in the sampling device according to the independent claim 5 and in the method according to the independent claim 12, as well as in the other claims. According to the invention, it relates, first of all, to a method for sampling body fluid of a living being by using a sampling device which obtains a sample that is led outside a skin or the like surface of a living being by a catheter, cannula or the like, the sample being led with a tube directly, or to be conveyed to the analysing device in order to at least partly define the contents of the body fluid. If a transfer pump suctions the sample brought-outside a skin or the like surface of a living being towards the analysing unit through a transfer tube to be taken to the analysing unit or to be examined otherwise and the sample being of a sufficient size, a sampling passage to a catheter, cannula or the like of the target to be examined, is closed with a shutter, after which at least one transport medium is led behind the sample, this arrangement enables automated sampling, in which the size of the sample can be made small, the sampling can be interrupted after a desired sample size in the transfer tube and transport medium forming its own sequence behind the sample can be led into the tube. Surface tension of the fluids in this connection in the tube having a diameter of about 3 mm is still often sufficient for holding the sample as whole at the fluid-gas separating surface.

If the said transfer pump pumps the transport medium that is behind into a waste reservoir, only sample, no other substances, is obtained to the analysing device.

If transport medum is led to the supply tube for the next sampling, a clear space, that is a sequence, is obtained, whereby the analysing device will get essentially only sample also from the next sample. If translucency of the sample obviously differs from the transport medium, the borderline of the sample and the transport medium can be monitored by a photocell.

If air or inert gas, as well as also liquid transport medium, is used as transport medium, can gaseous substance be used on both sides of the sample, and still, can transport fluid, the pumping characteristics of which usually are better than those of air or gas, be used on both sides of the sequence gas-sample-gas.

Since the analysing devices according to modern technology require a small sample size to be examined, typically of a size of 80-150 microlitres, can the tube that is used for transfer be measured quite small for its diameter. Thus, also air sequences on both sides of the sample can be quite small, for example like the sample in their volume, or slightly bigger, because the air or inert gas reaching the analysing unit does not at least remarkably impede the analysing process.

If the said transfer pump pumps the transfer fluid that is behind the sample to the waste reservoir, only the sample and some air that is before the sample and after the sample is attained at the analysing unit. The solution used only as pure transfer fluid can be separated by a photocell and a valve arrangement to the waste reservoir from which it has to be emptied to a sewer or the like.

If, for the next sampling, transfer fluid is brought from the transfer fluid reservoir to the supply tube, after which feeding of the transfer fluid to the supply tube is closed and air or inert gas is brought to the supply tube, a sampling sequence is obtained, in which, before the next sample sequence, there is an air sequence having a volume of e.g. 120 microlitres in the tube after the transfer fluid. Thus, sampling is carried out in the tube between the air sequences and there is transfer fluid on both sides of the sequence air-sample-air.

The invention relates also to a sampling device for defining by means of the analysing unit at least partly the contents of the body fluid of a living being, which sampling device comprises a catheter, cannula or the like, which is arranged for sampling to draw a sample, which is led with the tube to be conveyed to the said analysing unit. If the sampling device comprises a valve arrangement together with its chambers, from which there is a first aperture connection to said catheter, cannula or the like, a second aperture connection to the supply tube of the transport medium and a third opening to the transfer tube, through which the sample is led to the said transfer tube to be taken to the analysing device, a sampling device of the body fluid is obtained, by means of which automatically effected sampling is possible as well as fast and safe. Thus, there is no risk of getting the device obstructed, which could otherwise happen if body fluid is let stand in a small tube for some time. This could happen if all preventing measures are not properly performed in manually repeated sampling.

If a first position, in which the sample is led from the target to be examined to the analysing unit, can be chosen in the said valve arrangement, as well as at least a second position, in which the sample is not taken from the target to be examined to the analysing device, the size of sample could easily be adjusted suitable.

If the sampling device comprises means to lead at least one, but preferably two substances in form of fluid or gas to the said transfer tube, a pulse is attained, in which the body fluid sample is separated at its both ends.

If the substances to be led to the transfer tube can be varied by the said means, a portion containing air or inert gas can be formed in the tube on both sides of the body fluid sample, and transfer fluid sequences can be formed outside of this sequence.

If the said means comprise at least one valve by means of which the substance flow to be led to the transfer tube can be cut off, sequence forming can be well controlled.

If the sampling device comprises means by which it is possible to arrange an under pressure to the transfer tube, the under pressure can be used as a propulsive force for the substances in the tube.

If the sampling device comprises means by which under pressure can be adjusted, flow rates in the tube can be influenced by raising or lowering the under pressure.

The invention relates also to a method for using the sampling device for transferring the infusion fluid into a living being. If the sampling device, in an attached frame portion of the catheter, cannula or the like, has a connection passage via which infusion fluid is transferred to a living being while sampling is not being performed and while the infusion fluid flow is stanched elsewhere than to a living being via a catheter, cannula or the like, such an arrangement is very practical at least when values of the body fluid content have to be studied frequently, and at other times it is necessary to add infusion fluid. With the device according to the invention, it is also possible to have readiness to immediately start an important medication to an individual under observation as fluid infusion, for example in the incident of serious cardiac arrhythmia. A suitable division into periods can combine the functions performed with the device according to the invention into an operative arrangement, which has both sampling and analysing functions, as well as infusion function.

In the following, the invention is described in more detail by referring to the annexed drawing, in which

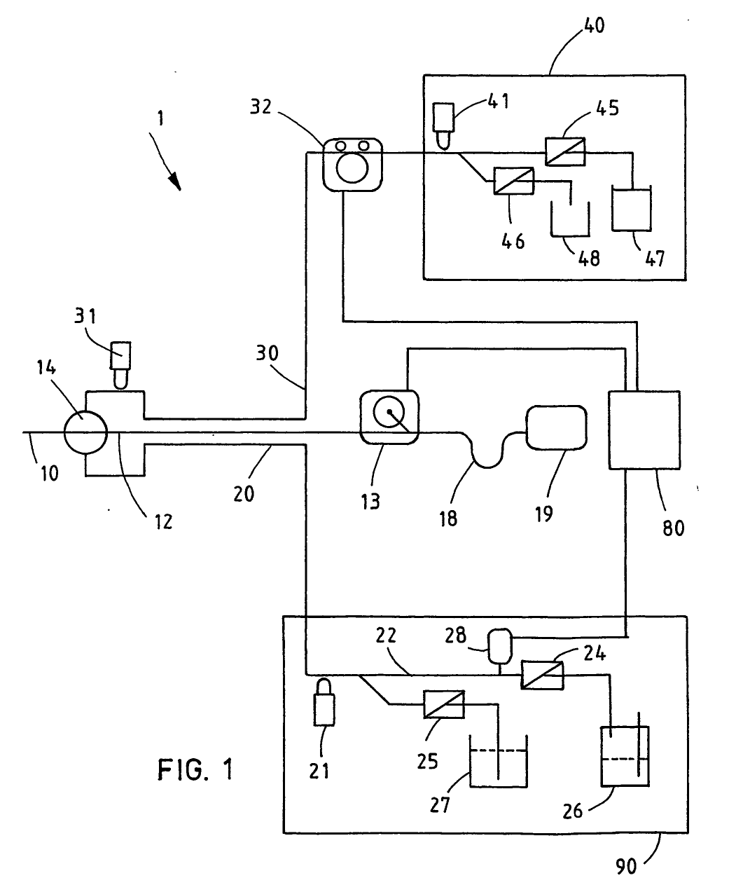

FIG. 1 shows schematically the sampling device according to the invention without infusion function,

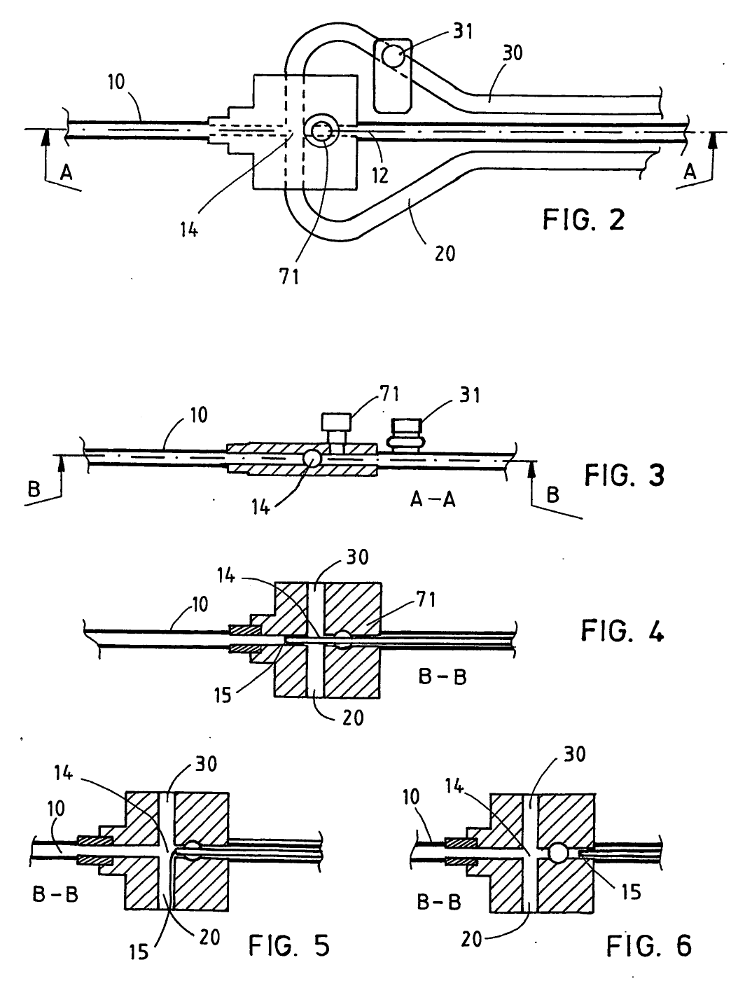

FIG. 2 shows schematically the valve arrangement according to the FIG. 1 together with its chambers as well as tubes arranged in the cannula frame and actuator connection,

FIG. 3 shows schematically the valve arrangement according to the FIG. 2 in direction A-A,

FIG. 4 shows schematically the valve arrangement according to the FIG. 3 cut in direction B-B and equipped with a closing piston which is in a closing position,

FIG. 5 shows schematically the valve arrangement according to the FIG. 4, in which the closing piston is in an opening position in relation to the sampling passage,

FIG. 6 shows schematically the valve arrangement according to the FIG. 4, which valve arrangement is in a position allowing the infusion fluid to travel,

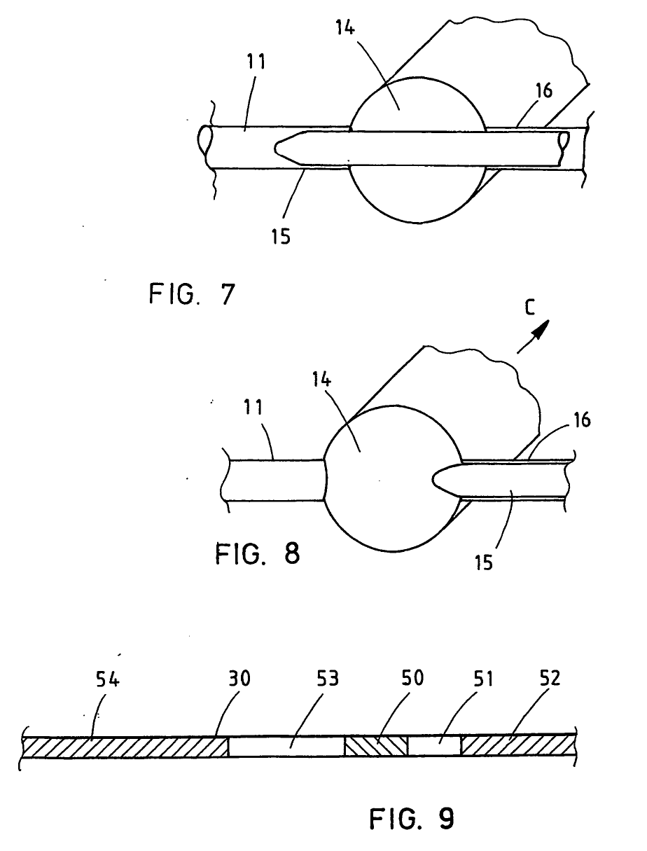

FIG. 7 shows schematically and enlarged the valve arrangement according to the FIG. 4 from the middle of the chamber, the closing piston being in a position that closes the sampling passage,

FIG. 8 shows schematically, similarly to the FIG. 7, the valve arrangement when the closing piston being in a position that opens the sampling passage and

FIG. 9 shows schematically the transfer tube between the chamber of the valve arrangement and the transfer pump, in which transfer tube the sample is between the air sequences and this sequence is between the transfer fluid sequences.

In the FIG. 1 of the drawing, the reference number 1 shows the sampling device according to the invention, in which Figure different parts are shown for the sake of clarity. Reference number 10 shows a connection to a catheter, cannula or the like connected to a living being and known as such, to which the sampling device 1 is connected. It is to note, that the invention does not relate to interior parts or arrangements of the skin or the like surface of a living being according to the method of the invention. The connection 10 has a chamber 14 and close to it a sensor 31, preferably a photocell that controls the substance flowing to the transfer tube 30 after the chamber 14. By means of the photocell, substances differing in their light permeability and their separating surfaces can be detected. A supply tube 20, via which, optionally liquid or gas flows to the chamber 14, is connected to the chamber 14. It is possible to use only air or inert gas as transport medium, that is, after the sample and before the sample, even though, by using both air or inert gas and liquid transport medium, a more reliable operation is attained. The supply tube 20 and the transfer tube 30 are, for example, of flexible plastic tube which is at least almost transparent and has an inner diameter of a rate of 1-4 mm, preferably about 3 mm. The structure of the connection 10 will be described below in more detail.

Reference number 90 shows a supply unit of the transport medium, in which supply unit there is a fluid reservoir 27 and a gas reservoir 26. Reference numbers 24 and 25 show valves of the substances led from these. Reference number 28 shows a sensor, preferably a pressure sensor, which is connected to a gas supply line 22 for controlling the pressure and for signalling the pressure information of the line to the control unit 80. The valve 25 is connected to the line for guiding the flow from the fluid reservoir 27 in the direction of the connection 10 and the valve has two positions—totally open and totally closed. The supply unit 90 includes also a sensor 21, preferably a photocell, which controls the fluid and gas flow in the supply tube 20. In order to clarify the matter, the connection of the sensor 21 to the control unit 80 is not described in the FIG. 1, even though such certainly does exist.

Sample separation equipment 40 includes the actual sample analysing unit 47, which is separated from the transfer tube 30 by means of the valve 45. Instead of the analysing unit 47, the sample can be taken e.g. to a microscope or be placed in a sample bottle or the like, if it is necessary to transport the samples to be examined elsewhere. Furthermore, there is a waste reservoir 48 to which the transfer fluid, transport fluid coming from the fluid reservoir 27 is collected. Valve 46 controls this flow. The equipment 40 includes further a sensor 41, which detects change of the substance, which has come via the transfer tube 30, to another.

The transfer pump 32, that is connected to the transfer tube 30 and that suctions the substance in the transfer tube 30 from the direction of the body connection 10 and pumps the substance led to the pump into the sample separation equipment 40, operates as a transport means. Control unit 80 controls the operation of the pump 32.

Control device 13 controls the closing function in connection with the chamber 14 of the valve arrangement in the manner shown below. Reference number 18 shows data transfer line, e.g. a fibre optic cable or the like that is connected to the data analyser 19. Data about the body fluid content and internal physiological conditions of the body obtained from the tip 15 can be transmitted along the data transfer line 18 to the data analyser 19, which is connected to a monitor or elsewhere (not shown) for transmitting data forward.

Even though different parts are shown in FIG. 1, most of the said functions can be assembled close to the catheter, cannula or the like. One possibility is to assemble the connection 10 close to the catheter, cannula or the like but to place other shown units e.g. about 2 meters away there from, but still very close to a living being, the body fluid of which is under observation. Usually blood properties of a living being, such as a human, animal or especially laboratory animal, are monitored, but also other body fluid, such as spinal fluid, urine, peritoneal fluid or other similar substances can be monitored. Also physical variables, such as pressure, can be monitored by means of a sensor (not shown) at the end 15 of a piston 12 when desired.

FIG. 2 shows in more detail parts and arrangements of the invention located close to the connection 10. The connection 10 is usually a tube made of plastic, through which a steel tube, which penetrates a skin or the like surface of a living being, is pushed at the beginning of insertion. This is known technique. In case a blood sample is taken of a target to be examined, the tip is directed to the blood vessel. The steel tube is pulled out and it is replaced by a closing piston 12 (not shown for the sake of clarity) described further in more detail, enfolded by the tube, and the tip 15 of which closing piston 12 functions near the chamber 14 in closing position. The closing piston can also be pushed further into the connection tube 10 according to need. The FIG. 2 also shows a connection of the supply tube 20 to the chamber 14, connection of the transfer tube 30 to the chamber 14 as well as the location of the sensor 31 in the transfer tube 30 when located some distance after the chamber 14 in the flow direction.

It is recommended that the length of the connection 10 is in a range of 32-45 mm and the distance of the first part of the connection 10 from the chamber 14 about 30 mm. The tip part of the connection 10 is, in the target to be examined, at a distance inside the skin or the like, e.g. in a blood vessel or elsewhere in the target to be examined, and the subcutaneous structures do not make part of the scope of the method according to the invention described.

FIG. 3 shows the structure shown in the previous FIG. 2 in direction A-A. The reference numbers are the same but it can be seen in FIG. 3, that the frame structure, in the middle part of which the chamber 14 is located, is thinnish, e.g. about 5-6 mm thick. The chamber 14 has an aperture of about 3 mm in between the tubes 20 and 30. Near the chamber 14 there is a fluid transfer connection 71 for supplying infusion fluid, but this is used only when needed. The fluid transfer connection 71 can be located elsewhere, too, such as in the tube 20 (not shown).

FIG. 4 shows a cut B-B, in which also the closing piston 12, which is moved to block the contact of the connection 10 to the chamber 14, can be seen. The diameter of the closing piston is of a category of 1 mm and the thickness of its arm portion to the control device 13 is of a category of 2 mm and preferably of flexible optical cable. Other reference numbers correspond to the parts described above.

FIG. 5 shows the closing piston 12 in a position allowing sample to flow via the connection 10 to the chamber 14. The closing piston blocks the infusion fluid flow via the connection 71 to the chamber. Other reference numbers correspond to the parts described above.

In the case shown in FIG. 6, the closing piston is pulled so far that the fluid flow via the connection 71 is led to the chamber 14 and further forwards via the connection 10. Flows via the connection 71 to the tubes 20 and 30 are blocked because the valves 24, 25, 45 and 46 shown in FIG. 1 are closed.

FIG. 7 shows enlarged the chamber 14 and structures connected to it. The closing piston 15 is at least somewhat coniform at its front part, so that the front part would meet the cannula tube 11 right at the wall of the chamber 14. The arm portion of the closing piston is on the other side of the chamber 14 in the tube 16. The gap is preferably quite short, e.g. 0.01-0.1 mm. The size of the closing piston is of a category of 1.2 mm and the diameter of the chamber about 3 mm.

FIG. 8 shows the case shown in FIG. 7, but the closing piston 12 is moved to a position in which the sample can flow from the cannula 11 to the chamber 14 and further in direction arrow C to the transfer tube 30.

FIG. 9 shows the transfer tube 30, in which the sample and other parts relating to it are in succession. Reference number 50 shows sample of which a length is in the tube 30. If the volume of the sample is of a category of 120 microliters, the length of a sample in a typical transfer tube is about 3 cm.

Before the sample 50 there is air or inert gas 51 preferably the same amount, that is 120 microliters and about 3 cm in length. Before this there is clearly more transfer fluid 52 from the reservoir 27 than the above-mentioned. Behind the sample 50 there is air or inert gas 53 somewhat more than 120 microliters, e.g. 200-300 microliters. Behind this there is, again, transfer fluid 54. Transfer fluid sequences 52 and 54 as well as the sample 50 between these and the separation airs 51 and 53 form a sample sequence.

From the sample sequence mentioned in the arrangement shown in FIG. 1, the transfer fluid sequences 52 and 54 are separated to the waste reservoir according to the data the sensor 41 sends to the control unit 80 the valve 46 being open and the valve 45 being closed. The sample 50 and parts of air 51 and 53 are led to the analysing device 47 of the sample. Usually such devices function faultlessly, even though air or inert gas partly reach their analysing means. Measuring data is sent to a patient monitor (not shown). After analysis, the sample 50 is led to the waste reservoir of the analysing device, which waste reservoir most usually is emptied to the waste (not shown).

The control unit 80 controls function of the device and controls the pump 32 as well as the valves 24, 25, 45 and 46 by acquiring its data from the sensors 21, 31 and 41 as well as from the sensor 28. The control unit 80 also controls function of the control device 13. All signal connections linked to the control unit 80 are not shown in the Figures for the sake of clarity. The course moving the shutter 12 of the control device 13 is at least about 20 mm but preferably clearly more, e.g. over 60 mm, so that the front part of the shutter 12 extends far into the body connection 10. Thus, with the closing piston, the tube can also be effectively prevented from being obstructed. It is recommended that the control unit be connected to a computer or the like arrangement, whereby by using Lab View® or other such suitable program the sampling events can be programmed to be taken e.g. every 10 minutes or every hour and also especially when needed, if the condition of the target to be examined suddenly deteriorates or in similar situations.

Even though the sampling device according to the invention does not feed, when analysing, transport fluid or other foreign substance to a patient via catheter, cannula or the like, it is necessary that all substances used are safe for the patient. The sampling device has also such a structure, that in case of malfunction, the resting positions of the components of the device are chosen so that the patient will not be in danger. This is solved, e.g. as far as the valves are concerned, by spring loaded magnet valves (not shown) that open only when the control unit 80 feeds them power.

1-12. (cancelled).

13 A method of sampling body fluid of a living being using a sampling device comprising, the method comprising:
drawing a sample of body fluid from a living being via a sampling passage and a transfer tube connected to a transfer pump;
closing the sampling passage by inserting a closing piston into the sampling passage when the sample reaches a desired size;
supplying a transport medium via a supply tube; and
supplying transport medium from the supply tube into the transfer tube behind the sample after the sampling passage is closed.

14 A method as in claim 13 wherein the transport medium behind the sample is pumped to a waste reservoir by the transfer pump.

15 A method as in claim 14 further comprising supplying transport medium to the supply tube for the next sampling.

16 A method as in claim 14 further comprising:
stopping flow of transport medium through the transfer tube;
stopping flow of transport medium through the supply tube; and
supplying infusion fluid into the sampling passage via a connection passage when the flow of transport medium through the transfer tube and the supply tube has been stopped but before the closing piston has been inserted into the sampling passage.

17 A sampling device for sampling body fluid of a living being, the sampling device comprising:
a chamber having first, second, and third aperture connections, the first aperture connection being connected to means for receiving body fluid from a living being;
a supply tube for supplying transport medium to the second aperture connection;
a transfer tube for transferring body fluid from the third aperture connection to an analyzing device; and
a closing piston which can be inserted into the first aperture connection to close the first aperture connection.

18 A sampling device as in claim 17 comprising a valve arrangement including the chamber, the valve arrangement having a first position, wherein a sample of body fluid can be supplied from the living being to the analysing device, and a second position, wherein the sample cannot be supplied from the living being to the analyzing device.

19 A sampling device as in claim 17 wherein the chamber comprises a passage connecting the second aperture connection to the third aperture connection, the closing piston being received through the chamber transversely of the passage but only partially obstructing the passage when the first aperture connection is closed.

20 A sampling device as in claim 19 wherein said passage and said closing piston are essentially cylindrical, the diameter of the passage being substantially larger than the diameter of the closing piston.

21 A sampling device as in claim 17 comprising means for supplying different transfer media to said chamber.

22 A sampling device as in claim 21 wherein the means for supplying different transport media comprise at least two lines connected to said supply tube, each said line having a respective valve for cutting off flow of medium in that line.

23 A sampling device as in claim 22 further comprising a transfer pump for drawing fluid into said transfer tube.

24 A sampling device as in claim 23 wherein the transfer pump is adjustable to control suction pressure.

25 A sampling device as in claim 17 further comprising:
a valve stopping flow of transport medium through the transfer tube;
a valve for stopping flow of transport medium through the supply tube; and
a connection passage for supplying infusion fluid into the first aperture connection when it is not closed by the closing piston.
